(19) United States
(12) Patent Application Publication
(10) Pub. No.: US 2026/0275336 A1
(43) Pub. Date: Sep. 17, 2026

(54) COMPOSITION FOR PREVENTING OR TREATING LUNG CANCER COMPRISING GCC2 INHIBITOR AS ACTIVE INGREDIENT

(71) Applicants: EXOPERT CORPORATION, Seoul (KR); KOREA UNIVERSITY RESEARCH AND BUSINESS FOUNDATION, Seoul (KR)

(72) Inventors: Sunghoi HONG, Seoul (KR); Hyesun JEONG, Hanam-si, Gyeonggi-do (KR); Yeonho CHOI, Seoul (KR)

(73) Assignees: EXOPERT CORPORATION, Seoul (KR); KOREA UNIVERSITY RESEARCH AND BUSINESS FOUNDATION, Seoul (KR)

(21) Appl. No.: 19/167,820

(22) PCT Filed: Mar. 27, 2023
(86) PCT No.: PCT/KR2023/004020

§ 371 Date: Sep. 22, 2025

(51) Int. Cl. C12N 15/113; A61P 35/00

(52) U.S. Cl. CPC C12N 15/113; A61P 35/00; C12N 2310/11; C12N 2310/315; C12N 2310/321; C12N 2310/3231; C12N 2310/531

(57) ABSTRACT

The present invention relates to a composition for preventing or treating lung cancer, comprising a GCC2 inhibitor as an active ingredient, and it has been confirmed through the present invention that the composition inhibits the expression of GCC2, which is secreted in a large amount in a lung cancer-specific manner, and thus can be used as a therapeutic agent for lung cancer, and as a result, the possibility of treating lung cancer was confirmed in vitro and in vivo by inducing invasion, metastasis, loss of the properties of mesenchymal cells, reduced tumorigenicity, and apoptosis of tumor cells.

### TECHNICAL FIELD

The present disclosure relates to a composition for preventing or treating a lung cancer, including a GRIP and coiled-coil domain-containing protein 2 (GCC2) inhibitor as an active ingredient.

### BACKGROUND ART

A tumor is an outcome of abnormal cell growth as cells lose their ability to control cell growth and death, and it is classified as a malignant tumor, or a cancer when the tumor has invasiveness, metastasis, and excessive proliferation. In addition to traditional treatment methods, there are immunotherapies and targeted therapies for treatment of the cancer. The targeted therapy refers to a treatment method that enables specific suppression of growth and development of a cancer using molecular therapeutics. Substances used for the targeted therapy include monoclonal antibodies and small molecules.

Antisense oligonucleotides are 10-20 bp long with an ability to complementarily bind to RNA in the nucleus and cytoplasm and suppress expression of target genes. Pre-mRNA is spliced prior to translation to form mRNA. Exons belong to mRNA, and introns belong to pre-mRNA, which is not included in mRNA. Antisense oligonucleotides bind to a splice site and interfere with mRNA formation. In addition, there are various compounds capable of regulating gene expression, and shRNA and siRNA are representative examples that are in use. Technologies for regulating gene expression such as shRNA and siRNA, including antisense oligonucleotides, involve complementary binding to mRNA to block a translation process.

When it comes to cancer treatment, development of more effective targeted therapies is required; however, to date, the development of targeted therapies that induce tumor suppression effects with an excellent therapeutic effect in vivo remains limited.

### DISCLOSURE OF THE INVENTION

### Technical Goals

Accordingly, an object of the present disclosure is to provide a composition for preventing, ameliorating, or treating a lung cancer, containing a GCC2 expression or activity inhibitor as an active ingredient.

### Technical Solutions

The present disclosure provides a pharmaceutical composition for preventing or treating a lung cancer, containing a GCC2 expression or activity inhibitor as an active ingredient.

In addition, the present disclosure provides a health functional food composition for preventing or ameliorating a lung cancer, containing a GCC2 expression or activity inhibitor as an active ingredient.

### Advantageous Effects

The present disclosure relates to a composition for preventing or treating a lung cancer including a GCC2 inhibitor as an active ingredient, wherein the present disclosure has revealed a probability as a lung cancer treatment agent by suppressing expression of GCC2, which is secreted in large amounts specifically in the lung cancer. As a result, the possibility in the lung cancer treatment was identified by inducing invasion, migration, loss of mesenchymal cell properties, decreased tumorigenicity, and apoptosis of tumor cells in vitro and in vivo.

### BRIEF DESCRIPTION OF DRAWINGS

FIG. 1 shows results of a scratch assay using shRNA of the present disclosure. It shows reduction in a cell migratability 24 hours later in an experimental group in which GCC2 expression was suppressed with shRNA unlike CD63-H1299, which is a control group.

FIG. 2 shows experimental results for a colony forming ability of cells whose gene expression is suppressed using shRNA of the present disclosure. After culturing for 7 days, as a result of calculating an average by counting colonies larger than 1 mm, it was found that, in case of an experimental group, half the number of colonies of a control group were formed.

FIG. 3 shows results of invasion and migration of cells transfected with shRNA of the present disclosure. It was revealed that the number of cells passing through a membrane decreased compared to a control group, with a significant decrease as in a statistical result.

FIG. 4 shows results of analyzing an apoptosis of cells transfected with shRNA of the present disclosure via a TUNEL assay. Compared to a control group, it may be noticed that cells expressing a green fluorescence are detected in an experimental group, indicating that apoptosis is taking place.

FIG. 5 shows a result of identifying tumor formation by subcutaneously injecting cells transfected with shRNA of the present disclosure and untransfected cells into mice. A tumor volume of the transfected cells reached 0 after 12 days, whereas a tumor size of a control group gradually increased.

FIG. 6 shows a result of an experiment conducted by subcutaneously injecting cells transfected with shRNA of the present disclosure and untransfected cells into mice and then sacrificing the mice when tumors in a control group reached 1 cm or greater. As a result of measuring a weight of extracted tumors, the tumors disappeared in all but one experimental group.

FIG. 7 shows a result of analyzing changes in a gene expression level when an antisense nucleotide of the present disclosure is transfected via Realtime PCR. It was determined that an expression level was suppressed when a specific antisense oligonucleotide was used.

FIG. 8 shows results of identifying an expression level by collected RNA at the same time after transfection by varying concentrations of antisense oligonucleotides of the present disclosure. It was determined that there are conditions for an appropriate concentration.

FIG. 9 shows results of identifying a difference in a GCC2 expression level over time by fixing a concentration of antisense oligonucleotides of the present disclosure. It was determined that an expression level recovered after a certain period of time.

FIG. 10 shows results of a case in which a modified antisense oligonucleotide of the present disclosure is transfected. It was found that an inhibition efficiency was excellent at a low concentration compared to that of FIG. 8.

FIG. 11 shows results of identifying a difference in an expression level by varying the time after transfection while fixing a concentration of antisense oligonucleotides modified in the present disclosure. As shown in FIG. 9, there was a difference in the expression level over time.

FIG. 12 shows results of identifying a residual ability within cells by tagging a modified antisense oligonucleotide of the present disclosure with fluorescent FAM. The antisense oligonucleotide was detected, which persisted at high levels for 3 days and remained within the cell for up to 10 days.

FIG. 13 shows results of identifying a scratch assay of cells transfected with a modified antisense oligonucleotide of the present disclosure. It was found that a cell migratability was reduced in an experimental group compared to a control group.

FIG. 14 shows results of identifying a colony forming ability of cells transfected with a modified antisense oligonucleotide of the present disclosure. It was found that the colony forming ability was reduced as shown in the drawing.

FIG. 15 shows results of identifying a migratabiltiy of cells transfected with a modified antisense oligonucleotide of the present disclosure. It was found that a migratabiltiy of cells was reduced in an experimental group compared to a control group.

FIG. 16 shows results of determining tumor formation by subcutaneously injecting cells transfected with a modified antisense oligonucleotide of the present disclosure and untransfected cells into mice. It was determined that a tumor size of an experimental group decreased from day 6 after cell transplantation.

FIG. 17 shows a result of an experiment conducted by extracting a tumor from mice subcutaneously transplanted with cells transfected with a modified antisense oligonucleotide of the present disclosure and untransfected cells when the tumor of a control group reached 1 cm or greater. As a result of measuring a weight, it was determined that an experimental group maintained a constant weight.

FIG. 18 shows a result of subcutaneously injecting PBS and a modified antisense oligonucleotide starting on day 8 after subcutaneous tumor transplantation into mice. As a result, it was found that a tumor forming rate was slowed in an experimental group compared to a control group.

FIG. 19 shows a result of an experiment conducted by extracting a tumor from a mice sacrificed when the tumor of a control group exceeded 1 cm in size, using mice subcutaneously injected with a modified antisense oligonucleotide of the present disclosure and mice injected with PBS. As a result of measuring a weight, two out of eight mice in a control group exceeded 400 mg, while all mice in an experimental group showed an average weight of 87 mg, demonstrating that tumor growth was suppressed.

### BEST MODE FOR CARRYING OUT THE INVENTION

The present disclosure provides a pharmaceutical composition for preventing or treating a lung cancer, containing a GCC2 expression or activity inhibitor as an active ingredient.

Specifically, the GCC2 expression inhibitor may be an antisense nucleotide, small interfering RNA (siRNA), and short hairpin RNA (shRNA) that complementarily bind to an mRNA of a GCC2 gene, and the GCC2 activity inhibitor may be a compound, peptide, peptide mimetics, aptamer, antibody, and natural product that specifically bind to a GCC2 protein, but are not limited thereto.

Preferably, the antisense nucleotide may be any one or more selected from nucleotide sequences represented by SEQ ID NO: 1 to SEQ ID NO: 26, but is not limited thereto.

Preferably, in the antisense oligonucleotide, each of one or more of nucleotides constituting the oligonucleotide may be 2′-O-methoxyethyl (2′-O-MOE) modified, or each of one or more of nucleotides may be a locked nucleic acid (LNA), or one or more of chemical bonds constituting a backbone of the nucleotide may be phosphorothioate (PS), but are not limited thereto.

More preferably, the antisense oligonucleotide may be modified by substituting three nucleotides at each of the 5′-terminal and 3′-terminal in the nucleotide sequences represented by SEQ ID NO: 1 to SEQ ID NO: 26 with locked nucleic acids (LNAs), substituting a central nucleotide with 2′-MOE, and linking each nucleotide by PS bond, but is not limited thereto.

More preferably, the antisense oligonucleotide represented by SEQ ID NO: 1 may bind to a target site represented by SEQ ID NO: 27 present in the GCC2 mRNA, the antisense oligonucleotide represented by SEQ ID NO: 2 may bind to a target site represented by SEQ ID NO: 28 present in the GCC2 mRNA, the antisense oligonucleotide represented by SEQ ID NO: 3 may bind to a target site represented by SEQ ID NO: 29 present in the GCC2 mRNA, the antisense oligonucleotide represented by SEQ ID NO: 4 may bind to a target site represented by SEQ ID NO: 30 present in the GCC2 mRNA, the antisense oligonucleotide represented by SEQ ID NO: 5 may bind to a target site represented by SEQ ID NO: 31 present in the GCC2 mRNA, the antisense oligonucleotide represented by SEQ ID NO: 6 may bind to a target site represented by SEQ ID NO: 32 present in the GCC2 mRNA, the antisense oligonucleotide represented by SEQ ID NO: 7 may bind to a target site represented by SEQ ID NO: 33 present in the GCC2 mRNA, the antisense oligonucleotide represented by SEQ ID NO: 8 may bind to a target site represented by SEQ ID NO: 34 present in the GCC2 mRNA, the antisense oligonucleotide represented by SEQ ID NO: 9 may bind to a target site represented by SEQ ID NO: 35 present in the GCC2 mRNA, the antisense oligonucleotide represented by SEQ ID NO: 10 may bind to a target site represented by SEQ ID NO: 36 present in the GCC2 mRNA, the antisense oligonucleotide represented by SEQ ID NO: 11 may bind to a target site represented by SEQ ID NO: 37 present in the GCC2 mRNA, the antisense oligonucleotide represented by SEQ ID NO: 12 may bind to a target site represented by SEQ ID NO: 38 present in the GCC2 mRNA, the antisense oligonucleotide represented by SEQ ID NO: 13 may bind to a target site represented by SEQ ID NO: 39 present in the GCC2 mRNA, the antisense oligonucleotide represented by SEQ ID NO: 14 may bind to a target site represented by SEQ ID NO: 40 present in the GCC2 mRNA, the antisense oligonucleotide represented by SEQ ID NO: 15 may bind to a target site represented by SEQ ID NO: 41 present in the GCC2 mRNA, the antisense oligonucleotide represented by SEQ ID NO: 16 may bind to a target site represented by SEQ ID NO: 42 present in the GCC2 mRNA, the antisense oligonucleotide represented by SEQ ID NO: 17 may bind to a target site represented by SEQ ID NO: 43 present in the GCC2 mRNA, the antisense oligonucleotide represented by SEQ ID NO: 18 may bind to a target site represented by SEQ ID NO: 44 present in the GCC2 mRNA, the antisense oligonucleotide represented by SEQ ID NO: 19 may bind to a target site represented by SEQ ID NO: 45 present in the GCC2 mRNA, the antisense oligonucleotide represented by SEQ ID NO: 20 may bind to a target site represented by SEQ ID NO: 46 present in the GCC2 mRNA, the antisense oligonucleotide represented by SEQ ID NO: 21 may bind to a target site represented by SEQ ID NO: 47 present in the GCC2 mRNA, the antisense oligonucleotide represented by SEQ ID NO: 22 may bind to a target site represented by SEQ ID NO: 48 present in the GCC2 mRNA, the antisense oligonucleotide represented by SEQ ID NO: 23 may bind to a target site represented by SEQ ID NO: 49 present in the GCC2 mRNA, the antisense oligonucleotide represented by SEQ ID NO: 24 may bind to a target site represented by SEQ ID NO: 50 present in the GCC2 mRNA, the antisense oligonucleotide represented by SEQ ID NO: 25 may bind to a target site represented by SEQ ID NO: 51 present in the GCC2 mRNA, and the antisense oligonucleotide represented by SEQ ID NO: 26 may bind to a target site represented by SEQ ID NO: 52 present in the GCC2 mRNA, but are not limited thereto.

More preferably, the shRNA may be any one or more shRNAs selected from the nucleotide sequences represented by SEQ ID NO: 53 to SEQ ID NO: 56, but is not limited thereto.

Preferably, the lung cancer may be, but is not limited to, a non-small cell lung cancer.

The pharmaceutical composition of the present disclosure may include chemicals, nucleotides, antisenses, siRNA oligonucleotides, and natural product extracts as an active ingredient. The pharmaceutical composition or complex preparation of the present disclosure may be prepared using a pharmaceutically suitable and physiologically acceptable adjuvant in addition to the active ingredient, and the adjuvant may include solubilizing agents such as excipients, disintegrants, sweeteners, binders, coating agents, swelling agents, lubricants, glidants, or flavoring agents. The pharmaceutical composition of the present disclosure may be preferably formulated as a pharmaceutical composition for administration by additionally including one or more types of pharmaceutically acceptable carriers in addition to the active ingredient. In the composition formulated as a liquid solution, acceptable pharmaceutical carriers include those that are sterile and biocompatible, such as saline solution, sterile water, Ringer's solution, buffered saline, albumin injection solution, dextrose solution, maltodextrin solution, glycerol, ethanol, and mixtures of one or more of these components, wherein other conventional additives, such as antioxidants, buffers, and bacteriostatic agents, may be added as necessary. In addition, diluents, dispersants, surfactants, binders, and lubricants may be additionally added to formulate into injectable formulations such as aqueous solutions, suspensions, and emulsions, pills, capsules, granules, or tablets.

The pharmaceutical formulation form of the pharmaceutical composition of the present disclosure may be a granule, powder, coated tablet, tablet, capsule, suppository, syrup, juice, suspension, emulsion, drop or injectable liquid, and a sustained-release formulation of the active compound. The pharmaceutical composition of the present disclosure may be administered in a conventional manner via intravenous, intraarterial, intraperitoneal, intramuscular, intraarterial, intraperitoneal, intrasternal, transdermal, intranasal, inhalation, topical, rectal, oral, intraocular, or intradermal routes. The effective amount of the active ingredient of the pharmaceutical composition of the present disclosure refers to an amount required for prevention or treatment of a disease. Therefore, it may be adjusted according to various factors including the type of a disease, severity of the disease, type and content of active ingredients and other ingredients contained in the composition, type of formulation, the patient's age, weight, general health status, sex and diet, administration time, administration route and secretion rate of the composition, treatment period, and concomitant drugs. Although not limited thereto, in the case of adults, for example, when administered once to several times a day, the composition of the present disclosure may be administered at a dosage of 0.1 ng/kg to 10 g/kg in the case of a compound, 0.1 ng/kg to 10 g/kg in the case of a polypeptide, protein, or antibody, and 0.01 ng/kg to 10 g/kg in the case of an antisense nucleotide, siRNA, shRNAi, or miRNA, when administered once to several times a day.

In addition, the present disclosure provides a health functional food composition for preventing or ameliorating a lung cancer, containing a GCC2 expression or activity inhibitor as an active ingredient.

Specifically, the GCC2 expression inhibitor may be an antisense nucleotide, small interfering RNA (siRNA), or short hairpin RNA (shRNA) that complementarily binds to the mRNA of the GCC2 gene, and the GCC2 activity inhibitor may be a compound, peptide, peptide mimetic, aptamer, antibody, or natural product that specifically binds to the GCC2 protein, but is not limited thereto.

Preferably, the antisense nucleotide may be any one or more selected from the nucleotide sequences represented by SEQ ID NO: 1 to SEQ ID NO: 26, but is not limited thereto.

Preferably, in the antisense oligonucleotide, each of one or more of nucleotides constituting the oligonucleotide may be 2′-O-methoxyethyl (2′-O-MOE) modified, or each of one or more of nucleotides may be a locked nucleic acid (LNA), or one or more of chemical bonds constituting a backbone of the nucleotide may be phosphorothioate (PS), but are not limited thereto.

More preferably, the antisense oligonucleotide may be modified by substituting three nucleotides at each of the 5′-terminal and 3′-terminal in the nucleotide sequences represented by SEQ ID NO: 1 to SEQ ID NO: 26 with locked nucleic acids (LNAs), substituting a central nucleotide with 2′-MOE, and linking each nucleotide by PS bond, but is not limited thereto.

Preferably, the lung cancer may be, but is not limited to, a non-small cell lung cancer.

The health functional food composition of the present disclosure may be provided in the form of a powder, granule, tablet, capsule, syrup, or beverage, and the health functional food composition may be used together with other foods or food additives in addition to the effective ingredient and may be used appropriately according to a conventional method. The amount of active ingredients mixed may be appropriately determined depending on its intended use, for example, preventive, health, or therapeutic treatment.

The effective dose of the active ingredient contained in the above health functional food composition may be used in accordance with the effective dose of the above pharmaceutical composition, but in the case of long-term intake for the purpose of health and hygiene or health control, it may be below the above range, and it is certain that the active ingredient may be used in an amount exceeding the above range because there is no problem in terms of safety.

There are no special restrictions on the types of the health foods, and examples include meat, sausage, bread, chocolate, candy, snacks, confectionery, pizza, ramen, other noodles, gum, dairy products including ice cream, various soups, beverages, tea, drinks, alcoholic beverages, and vitamin complexes.

Hereinafter, to help understanding of the present disclosure, example embodiments will be described in detail. However, the following examples are merely illustrative of the content of the present disclosure, and the scope of the present disclosure is not limited to the following examples. The examples of the present disclosure are provided to more completely explain the present disclosure to those skilled in the art.

### MODES FOR CARRYING OUT THE INVENTION

### <Example 1> In Vitro Inhibitory Effect Using shRNA

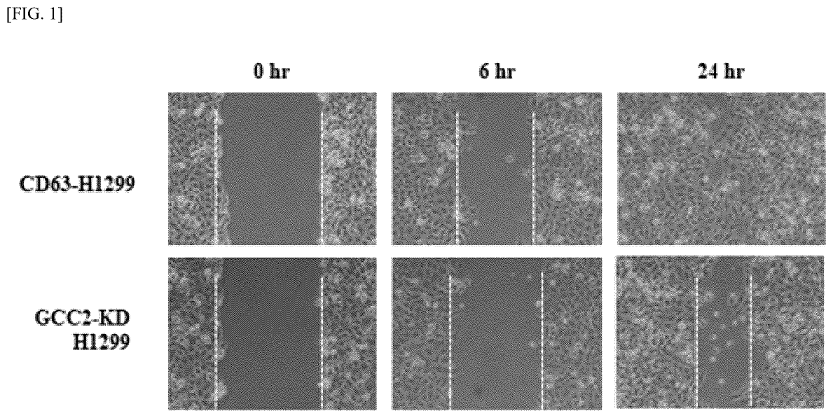

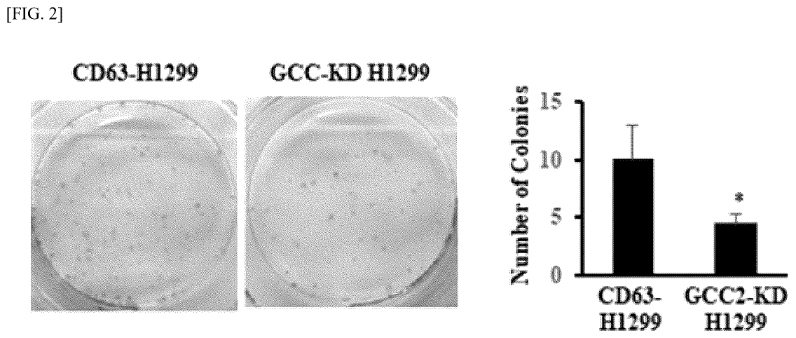

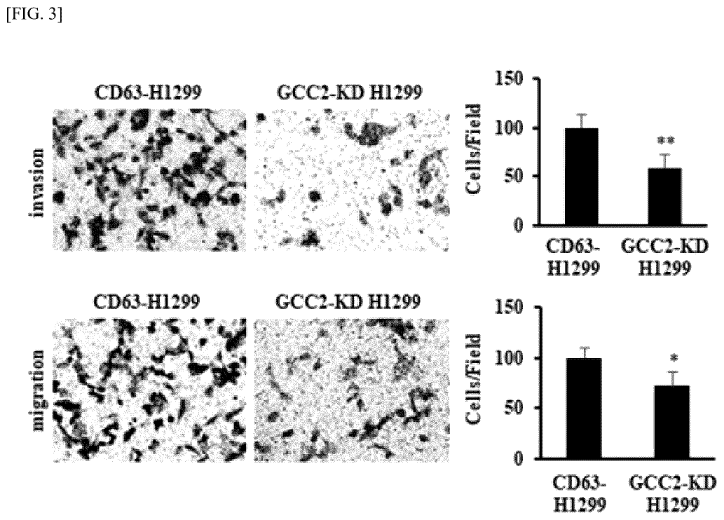

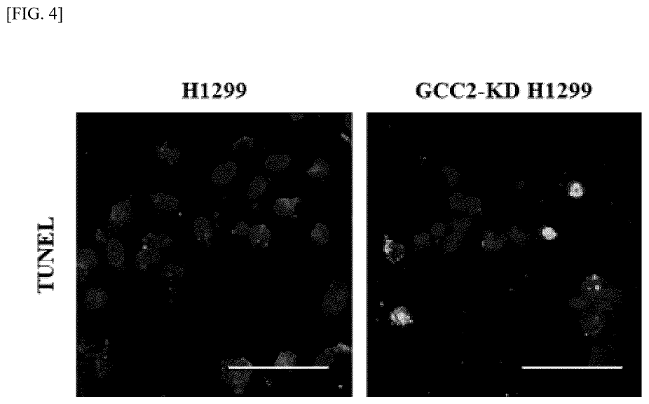

H1299 or H460 cells were cultured in a 35 mm dish at 1.2×10⁵ a day before. The next day, 4 ul of lipofectamine 2000 (Thermo fisher) and 8 μg of GCC2 shRNA plasmid (TRC lentiviral shRNA, horizon) were subjected to pretreatment according to the protocol provided by the manufacturer, and then the plasmid was delivered to the cells. Immediately after 5 hours of transfection, evaluation was carried out on colony forming ability and motility, as well as invasion/migration ability. To evaluate colony forming ability, transfected cells were seeded by 500-1,000 each into 35 mm dish or 6 well dish. After growing for up to ten days, the number of colonies formed of more than 100 cells was counted. Mobility evaluation was performed by scraping the bottom with a 200 ul tip to check a rate at which cells migrate into an empty space at regular time intervals. For invasion/migration evaluation, transfected cells were directly seeded into an upper chamber of Corning's Transwell for migration, and for invasion evaluation, cells were seeded after adding and hardening a solution of Matrigel and culture medium diluted in a ratio of 5:1 to 10:1. After 2 days, trypan blue staining was used to determine how much the seeded cells had moved to the bottom surface of the upper chamber. As a result, it was found that all of the above abilities related to the malignancy of cancer were reduced (FIGS. 1 to 4).

### <Example 2> Inhibitory Effect on Tumor Formation in an Animal Model of Cells in which GCC2 was Suppressed with shRNA

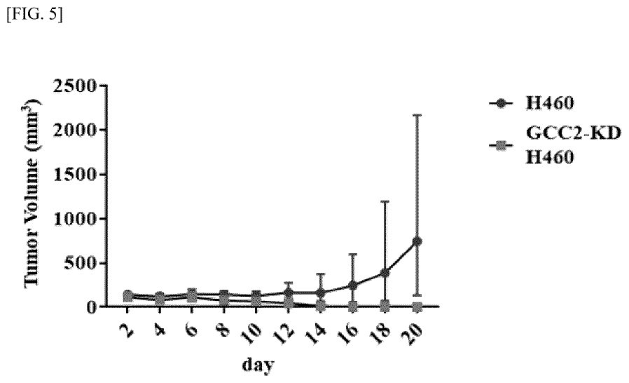

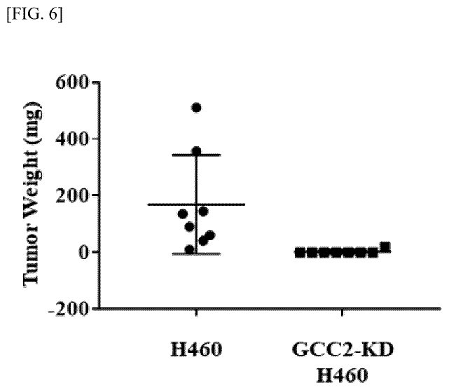

BALB/c nude mice were used for tumorigenesis experiments, and all 5-week-old males were used. After going through a one-week stabilization period, mice in the fourth week were injected subcutaneously with a mixture obtained by mixing 5×10⁶ H460 cells transfected as described in Example 1 with 50 ul of a 1:1 mixture solution of Matrigel and culture medium. Tumor growth was monitored at two-day intervals thereafter. As a result, it was determined that cancer cells lost their ability to form tumors (FIG. 5 and FIG. 6).

### <Example 3> Antisense Oligonucleotide Compounds Targeting GCC2

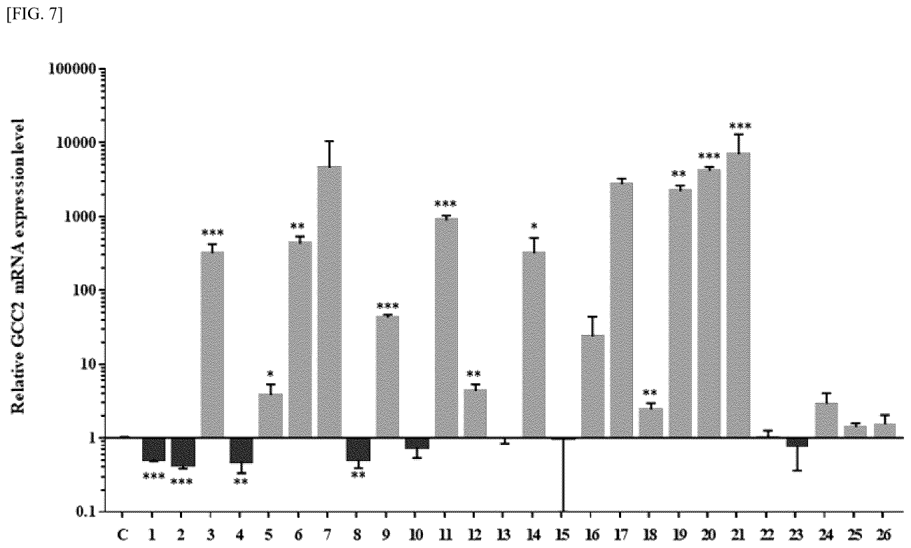

The oligonucleotides in Table 1 below were synthesized using standard techniques reported previously. In addition, among the modification technologies for antisense oligonucleotides developed so far, modification was carried out using locked nucleic acids (LNAs), 2′-O-methoxyethyl (2′-MOE), and phosphorothioate (PS). The modification involved substitution of the three sequences at the 5′ and 3′ terminal with LNA, the central nucleotide with 2′-MOE, and the internucleotide linkage with PS (FIG. 7).

TABLE 1

| # | ASO (5′ > 3′) |
|---|---|
| SEQ ID NO: 1 | CTCTTCACCTTAGAT |
| SEQ ID NO: 2 | CCCAATACCTGTACA |
| SEQ ID NO: 3 | TGATCTGAGTTCTTC |
| SEQ ID NO: 4 | GTGCATCTCCCATCT |
| SEQ ID NO: 5 | CTGCCATCAACTCATT |
| SEQ ID NO: 6 | CTTTTCATCAGTGGT |
| SEQ ID NO: 7 | GCATTTGAGGTGGCT |
| SEQ ID NO: 8 | GTCTTCCCGTTCATG |
| SEQ ID NO: 9 | TCTCTGTTATGCTGT |
| SEQ ID NO: 10 | GCTTCTTGCTTTTCT |
| SEQ ID NO: 11 | AAGCAGTTCTGAGCT |
| SEQ ID NO: 12 | GGTATCTCTTTGACT |
| SEQ ID NO: 13 | ACTACACTCTCTTCT |
| SEQ ID NO: 14 | GCAGGGCCTCTTTTT |
| SEQ ID NO: 15 | GTTCTAGTAGTGGCT |
| SEQ ID NO: 16 | CTTGTAAGGTCTTCA |
| SEQ ID NO: 17 | TTTTGTGCTTCTCTG |
| SEQ ID NO: 18 | GTTCCCTTTCTTGTT |
| SEQ ID NO: 19 | GTTCCGCCTCTTTGG |
| SEQ ID NO: 20 | CTCGTTCTGGGATGT |
| SEQ ID NO: 21 | GGTCGGTTCATTCTT |
| SEQ ID NO: 22 | GTCTGTGTTTCTCCT |
| SEQ ID NO: 23 | CTCTTCCCGGGTTAC |
| SEQ ID NO: 24 | GTGTGTATGTGCTGG |
| SEQ ID NO: 25 | GTGGTTTTATTCTGG |
| SEQ ID NO: 26 | TTCCAGTTGGTATGG |

### <Example 4> Concentration-Dependent Effects of Antisense Oligonucleotide Compounds In Vitro

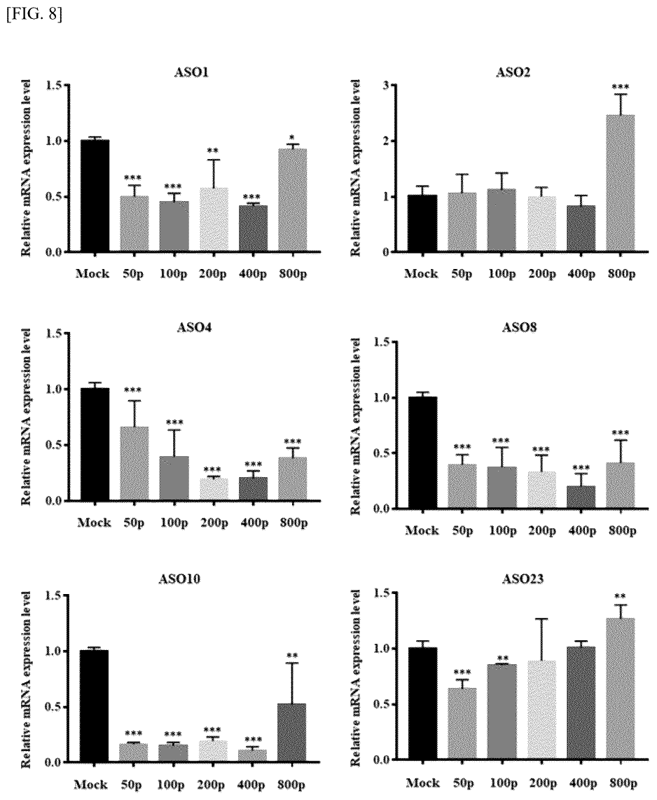

1.2×10⁵ H1299 cells were seeded into a 35 mm dish a day before. The next day, 50, 100, 200, 400, and 800 pmol of antisense oligonucleotides and 4 μl of lipofectamine 2000 were prepared according to the manufacturer's protocol to carry out transfection for 5 hours. Immediately after 5 hours of transfection, cells were subjected to RNA collection using Trizol (Thermo Fisher). The collected RNA was converted into cDNA by RT-PCR. cDNA was used to identify the expression level of GCC2 mRNA using Realtime PCR technology using sybr green (KAPA). As a result, it was determined that the gene expression inhibitory ability differed depending on the concentration, and stable expression inhibition was achieved at 400 pmol (FIG. 8).

### <Example 5> Time-Dependent Effects of Antisense Oligonucleotide Compounds In Vitro

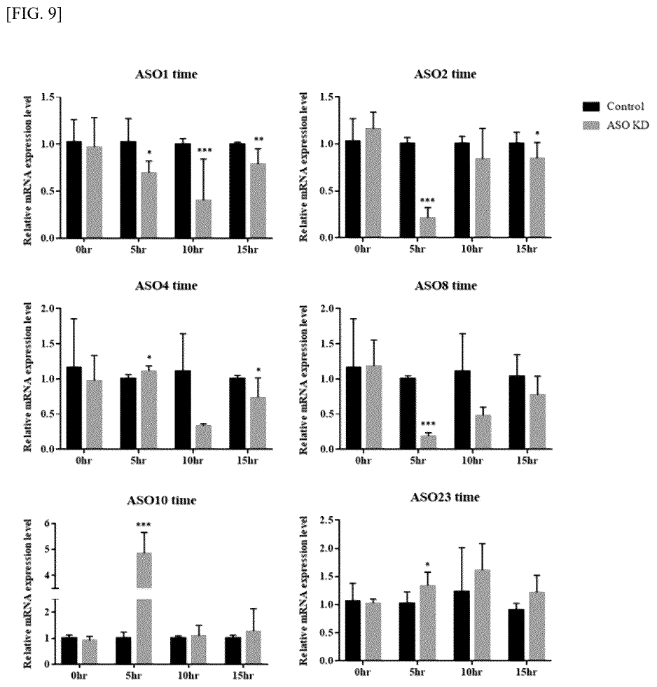

Cells were prepared in the same order as in Example 4 and transfected the next day using 400 pmol of antisense oligonucleotides and lipofectamine 2000. The GCC2 expression level was identified 5 hours, 10 hours, 15 hours, and 24 hours after the start of transfection (0 hours). The expression level was detected by Realtime PCR in the same manner as in Example 4. As a result, the time-dependent expression inhibitory effect was shown to be most effective at 5 hours (FIG. 9).

### <Example 6> Expression Inhibitory Effect of Modified Antisense Oligonucleotides

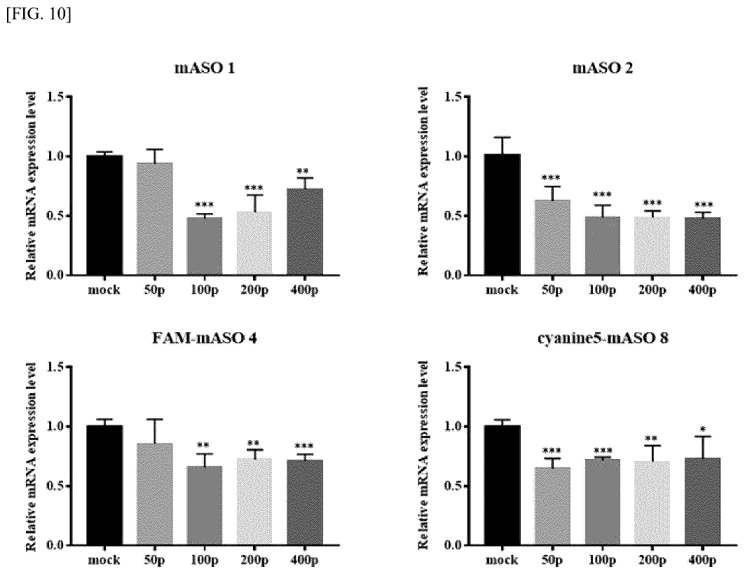

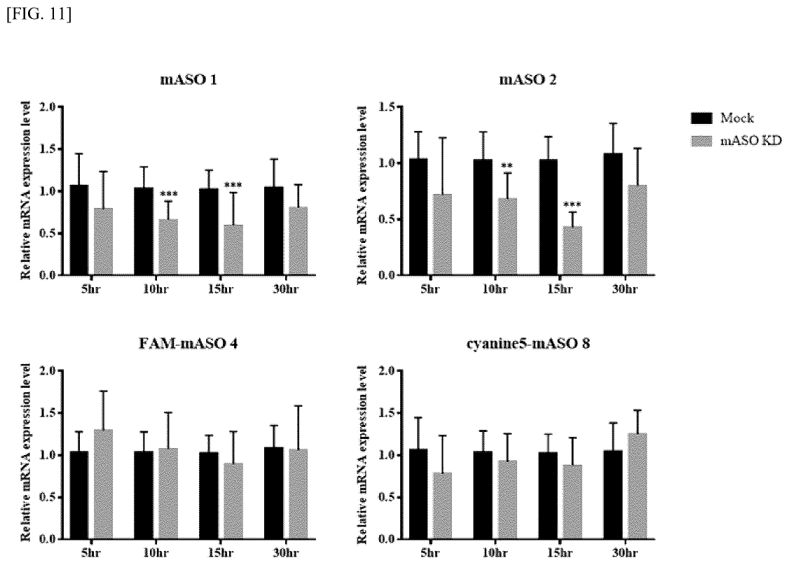

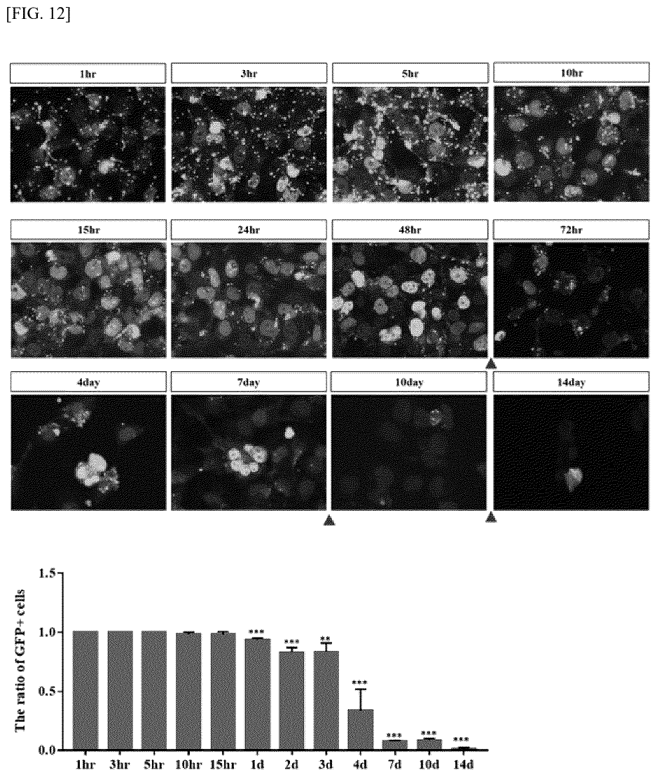

As described in Example 3, the most effective nucleotides were modified by combining LNA, 2′-MOE, and PS. The effectiveness was identified by conducting the same methods as in Examples 4 and 5. As a result, the modified form showed the better effect at a lower concentration of 100 μmol, while the most effective time was shown at 5 hours (FIGS. 10 to 12).

### <Example 7> In Vitro Effect of Antisense Oligonucleotide Compounds

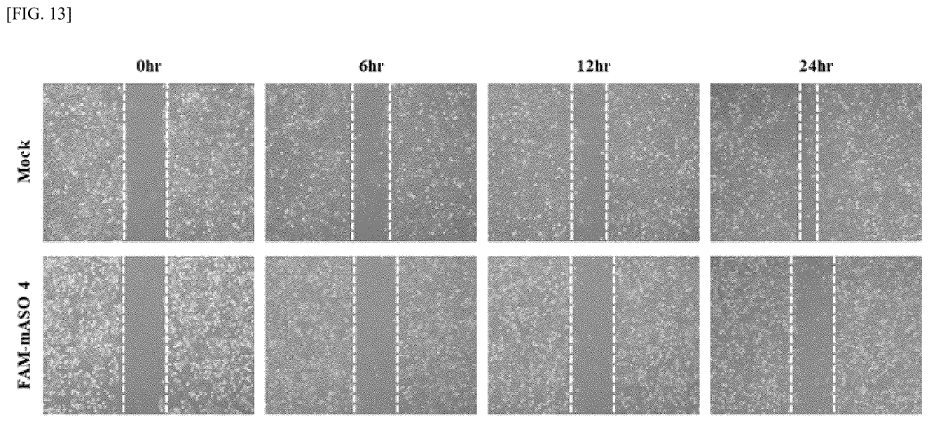

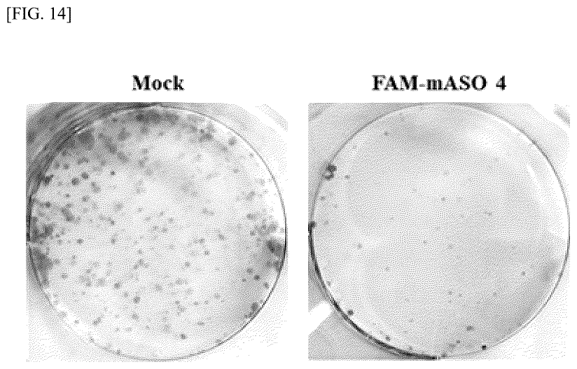

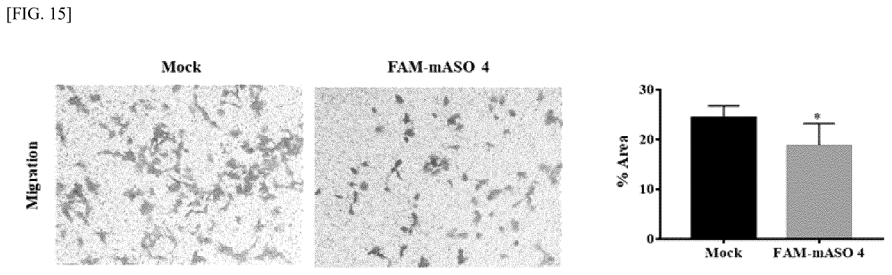

Proceeded in the same method as in Example 1, 100 pmol of the modified antisense oligonucleotide was used by mixing with 4 ul of lipofectamine 2000, respectively. As a result, it led to a decreased colony forming ability, decreased invasion/migration ability, and decreased mobility (FIGS. 13 to 15).

### <Example 8> Inhibitory Effect on Tumor Formation in an Animal Model of Cells in which GCC2 was Inhibited by an Antisense Oligonucleotide Compound

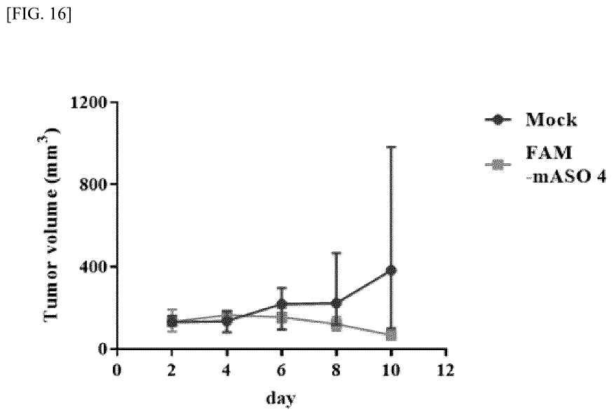

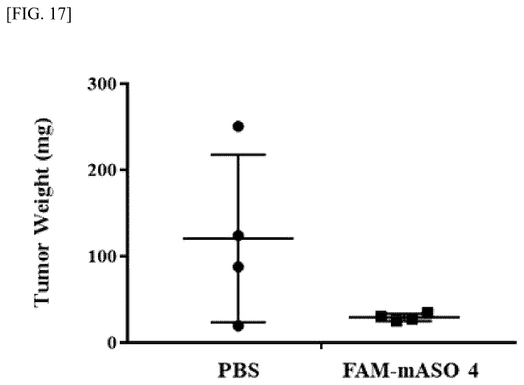

Proceeded in the same method as in Example 2, cells transfected with 100 pmol of the modified antisense oligonucleotide and lipofectamine 2000 were injected subcutaneously into BALB/c nude mice. As a result, it was found that the tumor size of the transfected cells decreased after day 6, whereas that of the H460 cells continued to increase (FIG. 16 and FIG. 17).

### <Example 9> Subcutaneous Injection of Antisense Oligonucleotide Compounds into Mouse Models

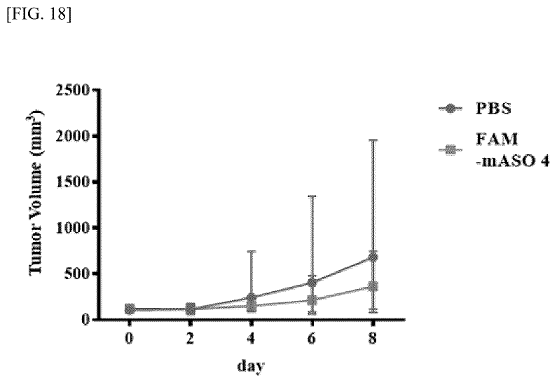

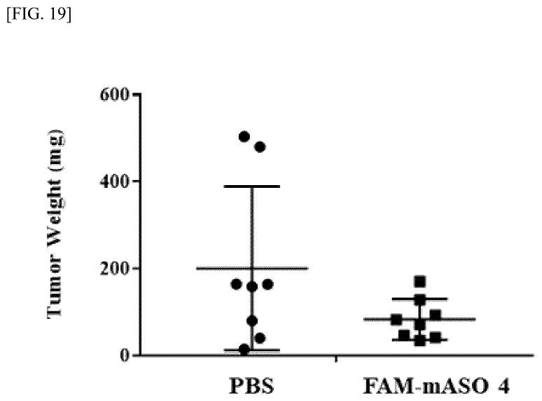

After stabilization for one week, 4-week-old male BALB/c nude mice were injected subcutaneously with 5×10⁶ H460 in combination with a Matrigel mixed solution. Eight days were allowed for the tumor to stably settle into place. From the day 8, the control group was injected subcutaneously with PBS, and the experimental group with the modified oligonucleotide at 5 mg/kg every 2 days. As a result, the difference in tumor size began to increase from the fourth day, and in the control group, two out of eight mice grew into large tumors measuring over 1 cm in diameter with a weight over 400 mg, while in the experimental group, all eight mice grew to an average of 83 mg. This indicates that delivery of antisense nucleotides via subcutaneous injection inhibits tumor growth (FIGS. 18 and 19).

Having described in detail a specific part of the contents of the present disclosure above, it is clear for those with ordinary skill in the art that this specific description is only a preferred example embodiment, and the scope of the present disclosure is not limited thereby. Thus, the substantial scope of the present disclosure is defined by the appended claims and their equivalents.

1. A method of preventing or treating a lung cancer, comprising:
administering a pharmaceutical composition comprising a GRIP and coiled-coil domain-containing protein 2 (GCC2) expression or activity inhibitor as an active ingredient to a subject.

2. The method of claim 1, wherein the GCC2 expression inhibitor is any one selected from the group consisting of an antisense nucleotide, small interfering RNA (siRNA), and short hairpin RNA (shRNA) that complementarily bind to an mRNA of a GCC2 gene.

3. The method of claim 1, wherein the GCC2 activity inhibitor is any one selected from the group consisting of a compound, peptide, peptide mimetic, aptamer, antibody, and natural product that specifically bind to a GCC2 protein.

4. The method of claim 2, wherein the antisense nucleotide is any one or more antisense oligonucleotides selected from nucleotide sequences represented by SEQ ID NO: 1 to SEQ ID NO: 26.

5. The method of claim 4, wherein, in the antisense oligonucleotide, each of one or more of nucleotides constituting the oligonucleotide is 2′-O-methoxyethyl (2′-O-MOE) modified, or each of one or more of nucleotides is a locked nucleic acid (LNA), or one or more of chemical bonds constituting a backbone of the nucleotide are phosphorothioate (PS).

6. The method of claim 5, wherein the antisense oligonucleotide is modified by substituting three nucleotides at each of the 5′-terminal and 3′-terminal in the nucleotide sequences represented by SEQ ID NO: 1 to SEQ ID NO: 26 with locked nucleic acids (LNAs), substituting a central nucleotide with 2′-MOE, and linking each nucleotide by PS bond.

7. The method of claim 4, wherein the antisense oligonucleotide represented by SEQ ID NO: 1 binds to a target site represented by SEQ ID NO: 27 present in the GCC2 mRNA, the antisense oligonucleotide represented by SEQ ID NO: 2 binds to a target site represented by SEQ ID NO: 28 present in the GCC2 mRNA, the antisense oligonucleotide represented by SEQ ID NO: 3 binds to a target site represented by SEQ ID NO: 29 present in the GCC2 mRNA, the antisense oligonucleotide represented by SEQ ID NO: 4 binds to a target site represented by SEQ ID NO: 30 present in the GCC2 mRNA, the antisense oligonucleotide represented by SEQ ID NO: 5 binds to a target site represented by SEQ ID NO: 31 present in the GCC2 mRNA, the antisense oligonucleotide represented by SEQ ID NO: 6 binds to a target site represented by SEQ ID NO: 32 present in the GCC2 mRNA, the antisense oligonucleotide represented by SEQ ID NO: 7 binds to a target site represented by SEQ ID NO: 33 present in the GCC2 mRNA, the antisense oligonucleotide represented by SEQ ID NO: 8 binds to a target site represented by SEQ ID NO: 34 present in the GCC2 mRNA, the antisense oligonucleotide represented by SEQ ID NO: 9 binds to a target site represented by SEQ ID NO: 35 present in the GCC2 mRNA, the antisense oligonucleotide represented by SEQ ID NO: 10 binds to a target site represented by SEQ ID NO: 36 present in the GCC2 mRNA, the antisense oligonucleotide represented by SEQ ID NO: 11 binds to a target site represented by SEQ ID NO: 37 present in the GCC2 mRNA, the antisense oligonucleotide represented by SEQ ID NO: 12 binds to a target site represented by SEQ ID NO: 38 present in the GCC2 mRNA, the antisense oligonucleotide represented by SEQ ID NO: 13 binds to a target site represented by SEQ ID NO: 39 present in the GCC2 mRNA, the antisense oligonucleotide represented by SEQ ID NO: 14 binds to a target site represented by SEQ ID NO: 40 present in the GCC2 mRNA, the antisense oligonucleotide represented by SEQ ID NO: 15 binds to a target site represented by SEQ ID NO: 41 present in the GCC2 mRNA, the antisense oligonucleotide represented by SEQ ID NO: 16 binds to a target site represented by SEQ ID NO: 42 present in the GCC2 mRNA, the antisense oligonucleotide represented by SEQ ID NO: 17 binds to a target site represented by SEQ ID NO: 43 present in the GCC2 mRNA, the antisense oligonucleotide represented by SEQ ID NO: 18 binds to a target site represented by SEQ ID NO: 44 present in the GCC2 mRNA, the antisense oligonucleotide represented by SEQ ID NO: 19 binds to a target site represented by SEQ ID NO: 45 present in the GCC2 mRNA, the antisense oligonucleotide represented by SEQ ID NO: 20 binds to a target site represented by SEQ ID NO: 46 present in the GCC2 mRNA, the antisense oligonucleotide represented by SEQ ID NO: 21 binds to a target site represented by SEQ ID NO: 47 present in the GCC2 mRNA, the antisense oligonucleotide represented by SEQ ID NO: 22 binds to a target site represented by SEQ ID NO: 48 present in the GCC2 mRNA, the antisense oligonucleotide represented by SEQ ID NO: 23 binds to a target site represented by SEQ ID NO: 49 present in the GCC2 mRNA, the antisense oligonucleotide represented by SEQ ID NO: 24 binds to a target site represented by SEQ ID NO: 50 present in the GCC2 mRNA, the antisense oligonucleotide represented by SEQ ID NO: 25 binds to a target site represented by SEQ ID NO: 51 present in the GCC2 mRNA, and the antisense oligonucleotide represented by SEQ ID NO: 26 binds to a target site represented by SEQ ID NO: 52 present in the GCC2 mRNA.

8. The method of claim 2, wherein the shRNA is any one or more shRNAs selected from nucleotide sequences represented by SEQ ID NO: 53 to SEQ ID NO: 56.

9. The method of claim 1, wherein the lung cancer is a non-small cell lung cancer.

10. A method of h preventing or ameliorating a lung cancer, comprising:
administering a health functional food composition comprising a GRIP and coiled-coil domain-containing protein 2 (GCC2) expression or activity inhibitor as an active ingredient to a subject.


SEQUENCE LISTING (56 sequences)

SEQ ID NO: 1 - DNA, 15 bp, synthetic construct
  source 1..15: mol_type = other DNA
ctcttcaccttagat

SEQ ID NO: 2 - DNA, 15 bp, synthetic construct
  source 1..15: mol_type = other DNA
cccaatacctgtaca

SEQ ID NO: 3 - DNA, 15 bp, synthetic construct
  source 1..15: mol_type = other DNA
tgatctgagttcttc

SEQ ID NO: 4 - DNA, 15 bp, synthetic construct
  source 1..15: mol_type = other DNA
gtgcatctcccatct

SEQ ID NO: 5 - DNA, 16 bp, synthetic construct
  source 1..16: mol_type = other DNA
ctgccatcaactcatt

SEQ ID NO: 6 - DNA, 15 bp, synthetic construct
  source 1..15: mol_type = other DNA
cttttcatcagtggt

SEQ ID NO: 7 - DNA, 15 bp, synthetic construct
  source 1..15: mol_type = other DNA
gcatttgaggtggct

SEQ ID NO: 8 - DNA, 15 bp, synthetic construct
  source 1..15: mol_type = other DNA
gtcttcccgttcatg

SEQ ID NO: 9 - DNA, 15 bp, synthetic construct
  source 1..15: mol_type = other DNA
tctctgttatgctgt

SEQ ID NO: 10 - DNA, 15 bp, synthetic construct
  source 1..15: mol_type = other DNA
gcttcttgcttttct

SEQ ID NO: 11 - DNA, 15 bp, synthetic construct
  source 1..15: mol_type = other DNA
aagcagttctgagct

SEQ ID NO: 12 - DNA, 15 bp, synthetic construct
  source 1..15: mol_type = other DNA
ggtatctctttgact

SEQ ID NO: 13 - DNA, 15 bp, synthetic construct
  source 1..15: mol_type = other DNA
actacactctcttct

SEQ ID NO: 14 - DNA, 15 bp, synthetic construct
  source 1..15: mol_type = other DNA
gcagggcctcttttt

SEQ ID NO: 15 - DNA, 15 bp, synthetic construct
  source 1..15: mol_type = other DNA
gttctagtagtggct

SEQ ID NO: 16 - DNA, 15 bp, synthetic construct
  source 1..15: mol_type = other DNA
cttgtaaggtcttca

SEQ ID NO: 17 - DNA, 15 bp, synthetic construct
  source 1..15: mol_type = other DNA
ttttgtgcttctctg

SEQ ID NO: 18 - DNA, 15 bp, synthetic construct
  source 1..15: mol_type = other DNA
gttccctttcttgtt

SEQ ID NO: 19 - DNA, 15 bp, synthetic construct
  source 1..15: mol_type = other DNA
gttccgcctctttgg

SEQ ID NO: 20 - DNA, 15 bp, synthetic construct
  source 1..15: mol_type = other DNA
ctcgttctgggatgt

SEQ ID NO: 21 - DNA, 15 bp, synthetic construct
  source 1..15: mol_type = other DNA
ggtcggttcattctt

SEQ ID NO: 22 - DNA, 15 bp, synthetic construct
  source 1..15: mol_type = other DNA
gtctgtgtttctcct

SEQ ID NO: 23 - DNA, 15 bp, synthetic construct
  source 1..15: mol_type = other DNA
ctcttcccgggttac

SEQ ID NO: 24 - DNA, 15 bp, synthetic construct
  source 1..15: mol_type = other DNA
gtgtgtatgtgctgg

SEQ ID NO: 25 - DNA, 15 bp, synthetic construct
  source 1..15: mol_type = other DNA
gtggttttattctgg

SEQ ID NO: 26 - DNA, 15 bp, synthetic construct
  source 1..15: mol_type = other DNA
ttccagttggtatgg

SEQ ID NO: 27 - DNA, 57 bp, Homo sapiens
  source 1..57: mol_type = genomic DNA
gatcttgttcaagatggggtggcttcaccagctacccctgggaccgggaaatctaag

SEQ ID NO: 28 - DNA, 25 bp, Homo sapiens
  source 1..25: mol_type = genomic DNA
cagaaagctaaatcaaggtgtacag

SEQ ID NO: 29 - DNA, 60 bp, Homo sapiens
  source 1..60: mol_type = genomic DNA
aattggagaaagaaattgaagaactcagatcaaaacctgttactgaaggaactggtgata

SEQ ID NO: 30 - DNA, 47 bp, Homo sapiens
  source 1..47: mol_type = genomic DNA
agatgggagatgcacataaggagttggaacaatcacatataaactat

SEQ ID NO: 31 - DNA, 36 bp, Homo sapiens
  source 1..36: mol_type = genomic DNA
aatgagttgatggcagtacgttccaaatacagtgaa

SEQ ID NO: 32 - DNA, 60 bp, Homo sapiens
  source 1..60: mol_type = genomic DNA
accactgatgaaaagaaggaaacagttactcaactccaaaatatcattgaggctaattct

SEQ ID NO: 33 - DNA, 10 bp, Homo sapiens
  source 1..10: mol_type = genomic DNA
agccacctca

SEQ ID NO: 34 - DNA, 30 bp, Homo sapiens
  source 1..30: mol_type = genomic DNA
catgaacgggaagacttagagtttaaaatt

SEQ ID NO: 35 - DNA, 22 bp, Homo sapiens
  source 1..22: mol_type = genomic DNA
acagcataacagagaagtacag

SEQ ID NO: 36 - DNA, 49 bp, Homo sapiens
  source 1..49: mol_type = genomic DNA
agaaaagcaagaagcaattttaaattatgagagtttacgagagattatg

SEQ ID NO: 37 - DNA, 17 bp, Homo sapiens
  source 1..17: mol_type = genomic DNA
agctcagaactgctttt

SEQ ID NO: 38 - DNA, 36 bp, Homo sapiens
  source 1..36: mol_type = genomic DNA
agtcaaagagataccatgttaaaagaattagaagga

SEQ ID NO: 39 - DNA, 25 bp, Homo sapiens
  source 1..25: mol_type = genomic DNA
agaagagagtgtagttcagtgtgaa

SEQ ID NO: 40 - DNA, 44 bp, Homo sapiens
  source 1..44: mol_type = genomic DNA
aaaaagaggccctgcagtctgatcttctagaaatgaagaatgct

SEQ ID NO: 41 - DNA, 60 bp, Homo sapiens
  source 1..60: mol_type = genomic DNA
aacctaaagccactactagaacaaaaagaattacgagataggagagcagagttgatacta

SEQ ID NO: 42 - DNA, 60 bp, Homo sapiens
  source 1..60: mol_type = genomic DNA
aataattttgagcatcgtattgaagaccttacaagacaattaagaaattcgactttgcag

SEQ ID NO: 43 - DNA, 38 bp, Homo sapiens
  source 1..38: mol_type = genomic DNA
cagagaagcacaaaatccacgagcacctgaaaacctct

SEQ ID NO: 44 - DNA, 60 bp, Homo sapiens
  source 1..60: mol_type = genomic DNA
aagaaaggtaaagtctgaattaaatatgcagattaaaactgtgtatttctgttttaggga

SEQ ID NO: 45 - DNA, 25 bp, Homo sapiens
  source 1..25: mol_type = genomic DNA
ccaaagaggcggaactccgggaaaa

SEQ ID NO: 46 - DNA, 53 bp, Homo sapiens
  source 1..53: mol_type = genomic DNA
acatcccagaacgaggtccttcgaaatagcttccgagatcaagtgcgacattt

SEQ ID NO: 47 - DNA, 22 bp, Homo sapiens
  source 1..22: mol_type = genomic DNA
aagaatgaaccgaccacaagaa

SEQ ID NO: 48 - DNA, 19 bp, Homo sapiens
  source 1..19: mol_type = genomic DNA
aggagaaacacagacctcc

SEQ ID NO: 49 - DNA, 40 bp, Homo sapiens
  source 1..40: mol_type = genomic DNA
gtaacccgggaagagggagaaggcatggagacaactgata

SEQ ID NO: 50 - DNA, 42 bp, Homo sapiens
  source 1..42: mol_type = genomic DNA
ccagcacatacacacagtctttagagcagctgcttaactctc

SEQ ID NO: 51 - DNA, 53 bp, Homo sapiens
  source 1..53: mol_type = genomic DNA
ccagaataaaaccaccaggaatgaattcactccccacttctctggaacctcag

SEQ ID NO: 52 - skipped

SEQ ID NO: 53 - RNA, 21 nt, synthetic construct
  source 1..21: mol_type = other RNA
ttaaccagctctaattcttgc

SEQ ID NO: 54 - RNA, 21 nt, synthetic construct
  source 1..21: mol_type = other RNA
aatagtatcaactctgctctc

SEQ ID NO: 55 - RNA, 21 nt, synthetic construct
  source 1..21: mol_type = other RNA
aactaaactcttcatgtcagc

SEQ ID NO: 56 - RNA, 21 nt, synthetic construct
  source 1..21: mol_type = other RNA
taaagcctaaattccagttgg